(19) United States
(12) Patent Application Publication
(10) Pub. No.: US 2003/0236538 A1
(43) Pub. Date: Dec. 25, 2003

(54) Artery and vein coupling tie string

(76) Inventor: Thomas Aikens, Tampa, FL (US)

Correspondence Address: THOMAS AIKENS, 3106 - E. LAKE AV, TAMPA, FL, 33610, US

(21) Appl. No.: 10/174,452

(22) Filed: Jun. 19, 2002

(51) Int. Cl. A61B 17/08

(52) U.S. Cl. 606/158; 606/151; 606/157

(57) ABSTRACT

The tie string is new to the field of embalming because there is no device used in the field of the funeral industry with self locking polymer teeth. There are linen, nylon, polyester, and cotton thread, non locking.

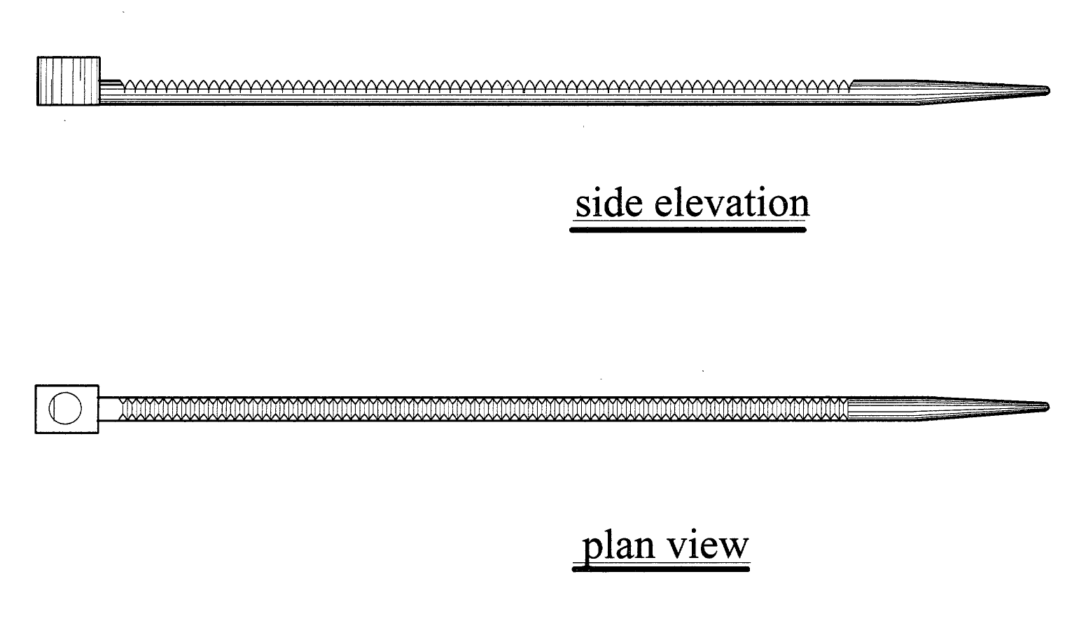

The title of this invention is Artery and Vein Coupling Tie String (A/V Coupling Tie String). The name of the inventor is Thomas Aikens, a citizen of the United States of America, residing at 3106 E. Lake Avenue, Tampa, Fla. 33610.

The tie strings we used with the arteries/veins coupling system of human remains after the embalming process. After the process of arterial injection of chemicals and draining of fluids by the embalmer, the tie strings are tied around both ring ends of the arterial/vein coupling. The tie strings are round with self locking teeth, adjustable throughout its entire length. The tie strings are made from one of the polymers; nylon, polycarbonate, polyethylene, and poly vinyl chloride. The tie strings are 8.00″, 9.00″, 16.00″, 24.00″ in length; 0.140″, 0.135″, 0.190″, 0.320″ in width.

### CROSS REFERENCE TO RELATED APPLICATIONS

Not Applicable

### STATEMENT REGARDING FEDERALLY SPONSORED RESEARCH DEVELOPMENT

Not Applicable

### REFERENCE TO MICROFICHE APPENDIX

Not Applicable

### BACKGROUND OF THE INVENTION

The field of use is the funeral industry, specifically in the embalming process of human remains. The subject matter of the claimed invention is to secure the arteries and veins coupling and vessels together after the process of embalming human remains. Using this invention will aide in solving the problem of leakage encountered by the embalmer. The only possible known related mechanism is in the construction field used for wrapping materials, flat tie strap.

### BRIEF SUMMARY OF THE INVENTION

The general idea of the claimed invention is to aide the arteries/veins coupling after the embalming process. Ensuring proper connection of the vessels to the arteries/veins coupling. This will solve the problem of the vessels slipping from the arteries and veins coupling.

### BRIEF DESCRIPTION OF THE SEVERAL VIEWS OF THE DRAWING

Not Applicable

### DETAILED DESCRIPTION OF THE INVENTION

The process of making the tie string is comprised of first a die of the tie string. Before a die is built, complete drawings and specifications of the tie string are required to guide the die maker. Each component must be measured and then fitted together, before testing for proper function. The die is a tool used in conjunction with an injection molding machine. The injection molding machine processes polymers of the thermoplastic material which is fed into a hopper, that is attached on the top of the machine and emerges as hot melt at the die. This process consists of heating and homogenizing plastic granules or polymers in a cylinder until there is sufficient fluid to allow for pressure injection into a relatively cold mold where they solidify and take the shape of the mold cavity or die. In the process of embalming, which is chemically treating the dead human body to reduce the presence and growth of microorganisms, to retard organic decomposition and to restore life-like appearance. The arteries and veins are used for this process by making an incision in both vessels and chemically treating the human remains. After this technique the A/V Coupling would be inserted in both artery and vein with the tie strings on both ends to prevent leakage of fluids and pressure build up in the body, which allows equal pressure to all parts of the human remains, this will keep swelling from occurring. There is no other product on the market in the funeral industry to compare with the tie string. The entire tie string is made from polymer. Polymer is a chemical reaction in which a number of similar small molecules is called polymerization, and the macromolecular product is a polymer.

In the human body, blood is contained within many tube like structures, the arteries and veins which compose the vascular system. The blood is forced to flow continuously to all parts of the body by the powerful pumping action of the heart. After death, the heart no longer can perform this duty, the use of a embalming machine is used. The embalming machine acts as the heart, which creates pressure in the body that force the blood out of the arteries and veins. An embalming instrument is placed in both artery and vein to remove blood from the dead human remains. The removal of the blood from the arteries and veins, the embalmer must make an incision in both arteries and veins. The arteries/vein coupling will come in place at this time and be inserted in both vessels with the guide director. Once the arteries and veins coupling are inserted, then the tie strings are placed around both ring ends of the coupling.

1. What I claim as my invention is that the round tie string will be placed around both ring ends of arteries/vein coupling, keeping it from slipping which it will prevent leakage of embalming fluid.

2. What I claim as my invention is that the tie strings will prevent pressure build up because of its interlocking process, which lock both ends of the coupling to allow continuous flow of fluids.
